(19) United States
(12) Patent Application Publication
(10) Pub. No.: US 2013/0178389 A1
(43) Pub. Date: Jul. 11, 2013

(54) COMPOSITE ASSAY FOR DEVELOPMENTAL DISORDERS

(71) Applicants: Stanley N. Lapidus, Bedford, NH (US); Stanley Letovsky, Milton, MA (US)

(72) Inventors: Stanley N. Lapidus, Bedford, NH (US); Stanley Letovsky, Milton, MA (US)

(21) Appl. No.: 13/735,435

(22) Filed: Jan. 7, 2013

Related U.S. Application Data
(60) Provisional application No. 61/583,699, filed on Jan. 6, 2012.

(51) Int. Cl. C12Q 1/68

(52) U.S. Cl. CPC C12Q 1/6883
USPC 506/9; 435/6.11

(57) ABSTRACT

This invention relates generally to diagnosing developmental disorders by detecting two or more genetic characteristics from a nucleic acid extracted from a sample taken from a patient. The genetic characteristics detected include nucleic acid expression profiles, nucleic acid sequences, and nucleic acid copy numbers. The genetic characteristics may be detected using sequencing technology, array based technology, or both. At least two genetic characteristics are compared to respective controls. From the comparison a diagnostic profile of a developmental disorder for the patient is formed.

### RELATED APPLICATION

The present patent application claims the benefit of and priority to U.S. Provisional Patent Application Ser. No. 61/583,699, filed on Jan. 6, 2012, the entirety of which is herein incorporated by reference.

### FIELD OF THE INVENTION

This invention relates generally to diagnosing autism and other developmental disorders.

### BACKGROUND INFORMATION

Autism and other developmental disorders disrupt the normal development of children and are estimated to affect 1 in 110 children. Developmental disorders may include mental disabilities, physical disabilities, or both. Typically, developmental disorders are diagnosed by observing and assessing a child's behavior, including an assessment of the child's cognitive and communicative functions. Although clinical evaluations are a useful tool in assessing a child's developmental delay, such evaluations are limited because a child's behavior is often transient and a child might not be exhibiting diagnostic behavior oddities on the day of the evaluation. Further, the evaluations often fail to indentify the specific cause of the delay. Thus, clinical evaluations often fail to provide a definitive diagnosis of a developmental disorder. Due to this lack of a definitive diagnosis, the genetic basis of the developmental disorder is being utilized to help indentify the specific cause of the developmental delay and to provide a more objective diagnosis of the developmental disorder than the behavioral evaluation.

Developmental disorders have been linked to genetic characteristics, including variations in nucleic acid expression profiles, nucleic acid sequence, and nucleic copy number. While these genetic indicia have associational value, they are not alone predictive of a disorder. For example, copy number alone appears not to be informative for autism spectrum disorder. Moreover, expression data are uninformative for some 50% of children suspected to have a developmental disorder. As a result, monolithic tests for developmental disorders fail to either accurately diagnose or accurately stage a disorder once diagnosed. Thus, new methods are needed to accurately diagnose and stage the severity of developmental disorders.

### SUMMARY

The invention provides methods for assessing a cognitive disorder by taking into account underlying genetic information as well as gene expression data. Methods of the invention result in improved ability to diagnose the presence of a disorder as well as the ability to distinguish between developmental disorders.

Methods of the invention recognize that a single genetic marker type is insufficient to diagnose and characterize developmental disorders with high sensitivity and specificity. According to the invention, methods that comprise multimodal analysis have greater sensitivity and specificity in the diagnosis and characterization of cognitive disorders.

Methods of the invention involve conducting an assay to measure a DNA characteristic in a sample obtained from a patient and conducting an assay on an RNA characteristic in that same sample. The obtained measures are used to diagnose a cognitive disorder. The DNA characteristic can be any measure of DNA, such as copy number, mutations, single nucleotide polymorphisms, or large-scale polymorphisms. The primary RNA characteristic is expression in terms of the amount of expression from a particular gene or genes and the particular RNA that is expressed. The invention also contemplates the use of micro RNA and small interfering RNA.

The invention also contemplates methods for classifying patients suspected of having a cognitive disorder by conducting an assay of a genomic change together with an assay for a change in the expression level of at least one gene by, in each case, comparison to levels observed in a population of patients known not to have a cognitive disorder. As above, the genomic change may be any genomic change (e.g., mutations, polymorphisms, rearrangements, deletions, insertions, alterations of methylation status and the like) and may be measured using array technology, sequencing, hybrid capture, and other known techniques.

The invention is also useful in combing nucleic acid and protein information in order to improve diagnostic sensitivity and specificity. Proteins are measured using known techniques, including but not limited to sequencing, chromatography (e.g., Western Blots), mass spectrometry and others. Protein and nucleic acid markers are measured and compared to standards indicative of disease or no disease, as with the nucleic acid measurements described above.

In accordance to the invention, a sample is obtained from a patient for testing. The sample may be any body fluid or tissue, such as blood, check swab, hair, skin, saliva, sputum, urine and the like. Nucleic acid and/or protein is extracted from the sample by well-known means. The extracted nucleic acid or protein is then characterized with respect to markers (either specific genes or expression products or quantitative markers, such as copy number and expression profiling) known to be associated with cognitive developmental disorders. Characterization can be by sequencing (which may be whole genome or whole protein sequence determination or may be directed at portions of the genome or proteome suspected or known to be associated with one or more cognitive developmental disorders), capture (e.g., hybrid capture or chromatography) or other known methods for characterizing nucleic acids and proteins. With respect to nucleic acids, the invention contemplates a combination of genomic analysis (e.g., mutations, single nucleotide polymorphisms and the like) and expression analysis. The invention also contemplates combining nucleic acid and protein markers, such as genotyping, expression analysis, amount of protein and the like.

Combinations of genomic and phenotypic markers are assessed in methods of the invention. Levels of various biomarkers are determined by methods known in the art and are compared to levels expected to be obtained in either samples from non-affected patients or samples from affected patients, depending on the desired diagnostic. Reference samples may be obtained empirically from healthy individuals or affected individuals; or may be obtained from a database.

Methods of the invention are useful for diagnosing cognitive disorders and, in particular, developmental disorders, including autism spectrum disorders, Angelman syndrome, cerebral palsy, Aspergers syndrome, Pervasive Developmental Disorder not otherwise specified (atypical autism), Childhood Disintegrative Disorder, Cohen syndrome, Down syndrome, Fragile X syndrome, IsoDicentric 15, Jacobsen syndrome, Prader-Willi syndrome, Rett syndrome, Coffin-Lowry syndrome, Williams syndrome, and Cornelia de Lange syndrome.

### DETAILED DESCRIPTION

Methods of the invention provide a sensitive and specific test for cognitive disorders, especially developmental cognitive disorders. The invention recognizes that genomic information alone may be insufficient for diagnosis and classification of cognitive disorders. Rather, genomic information supplemented by other markers, such as expression profiling and protein analysis, provides a much more robust analysis tool. In one aspect the invention addresses developmental cognitive disorders. Based upon traditional behavioral analysis, approximately 8.5% of children have some type of developmental disorder. However, it is estimated that only about 1% of those are properly placed on the autism spectrum. Treatment can be highly-effective if directed properly and the proper direction of treatment depends upon effective diagnostic and classification tools. Behavioral analysis is not sufficiently sensitive and specific to properly classify the majority of affected individuals. Genomic analysis, usually in the form of analysis of mutational and polymorphic variants, is also not specific and sensitive. Finally, expression analysis alone fails to capture the full scope of diagnosis and classification. It is a combination of different types of analysis (e.g., genomic, proteomic, expression) that provides the discriminatory power necessary to properly diagnose and classify patients on the spectrum of developmental disorders.

Methods of the invention rely on multiple markers of different types in order to achieve superior diagnostic accuracy. In one embodiment, a DNA assay is combined with an RNA assay. A negative DNA assay alone is not predictive because traditional DNA assays have a high false negative rate. In combination with a confirmatory RNA assay (e.g., expression analysis), the desired high negative and positive predictive values are achieved. In general, the invention provides information on the biological consequences of genomic changes in order to inform a diagnosis or classification. For example, a change in expression or in protein concentration may be indicative of an underlying, and sometimes undetected, change in the genome. To the extent that genomic changes are not predictive, changes in RNA expression or in proteins (either the array of proteins produced or the amount of protein produced) provide the information required for accurate diagnosis and classification.

Accordingly, methods of the invention provide for a evaluating a patient sample for any combination of two or more characteristics in order to form a more complete diagnostic profile for cognitive disorders.

### Obtaining a Biological Sample

Methods of the invention involve obtaining a sample, e.g., cell, tissue, blood, bone, or body fluid. Samples may include blood, a blood fraction, saliva, sputum, urine, semen, transvaginal fluid, cerebrospinal fluid, or stool. Other such samples may include tissue from brain, kidney, liver, pancreas, bone, skin, eye, muscle, intestine, ovary, prostate, vagina, cervix, uterus, esophagus, stomach, bone marrow, and lymph node.

The sample may be obtained by methods known in the art, such as a cheek swab, phlebotomy, fine needle aspiration, core needle biopsy, vacuum assisted biopsy, direct and frontal lobe biopsy, shave biopsy, punch biopsy, excisional biopsy, or cutterage biopsy.

Once the sample is obtained, nucleic acids are extracted to assess nucleic acid expression profile, nucleic acid sequence, and nucleic acid copy number. Certain aspects of the invention provide for drawing a blood sample and dividing the blood sample into two tubes, one for DNA analysis and the other for RNA analysis. Preferably enough blood is drawn to fill both tubes. The invention also provides for obtaining different sample types for either RNA analysis or DNA analysis. For example, the sample used for DNA analysis may be taken from a cheek swap, while the sample for RNA analysis may be taken from a blood draw.

### Nucleic Acids

Nucleic acids may be obtained by methods known in the art. Generally, nucleic acids can be extracted from a biological sample by a variety of techniques such as those described by Maniatis, et al., Molecular Cloning: A Laboratory Manual, Cold Spring Harbor, N.Y., pp. 280-281, (1982), the contents of which is incorporated by reference herein in its entirety.

It may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl₃ to denature any contaminating and potentially interfering proteins.

Methods of the invention also provide for isolation of mRNA from a target sample. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of theses references is incorporated by reference herein in their entirety. In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

### Detection

After extraction, various methods and combination of techniques such as sequencing and array based technologies may be utilized in methods of the invention in order to determine the nucleic acid expression, nucleic acid sequence and nucleic acid copy number. Nucleic acids include deoxyribonucleic acid (DNA) or ribonucleic acid (RNA). DNA, RNA, and copy number may be detected using a variety of sequencing and array based techniques.

Embodiments of the invention provide for whole genome sequencing, whole exome sequencing, whole transcriptome sequencing, RNA sequencing, DNA sequencing, or targeted sequencing of one or more specific genes indicative of the developmental disorder, such as single nucleotide polymorphism sequencing. Utilizing the above sequencing techniques allows for comprehensive sequencing of the sample or targeted sequencing of the sample. In comprehensive sequencing, such as whole genome sequencing or whole transcriptome sequencing, the entire DNA or RNA structure is examined. In targeted sequencing techniques, only target portions of the DNA or RNA are sequenced.

Whole genome sequencing determines the complete DNA sequence of the genome at one time. Whole genome sequencing covers sequencing of almost 100 percent, usually around 95%, of the sample's genome. Whole exome sequencing is selective sequencing of coding regions of the DNA genome. The targeted exome is usually the portion of the DNA that translate into proteins, however regions of the exome that do not translate into proteins may also be included within the sequence. Also, the targeted exome may be chosen because genes within the exome are known to causally relate to autism or other developmental disorders. The invention also provides for comprehensive and targeted RNA expression detection. For example, the invention provides for detection via whole transciptome sequencing or amplification. Whole transcriptome sequencing or amplification allows one to determine the expression of all RNA molecules including messenger RNA (mRNA), ribosomal RNA (rRNA), transfer RNA (tRNA), and non-coding RNA. Targeted RNA sequencing or amplification captures sequences of RNA from a relevant subset of a transcriptome in order to view high interest genes, i.e. those suspected of being causally linked to autism and/or other developmental disorders.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslavsky, et al., PNAS (USA), 100: 3960-3964 (2003), the contents of each of these references is incorporated by reference herein in its entirety.

An RNA sequence can also be detected by single molecule sequencing such as in Helicos Direct RNA sequencing method. Fatih Ozsolak, et al., Direct RNA sequencing. Nature 461, 814-818. Total RNA or RNA fragments with natural polyA tails are introduced to poly(dT) coated flow cells in order to enable capture and sequencing of polyA RNA species. In situations where the RNA does not have a polyA tail, for example small sample species, a polyA polymerase is introduced to the RNA in order to generate a polyA tail so that the sample RNA may attach to the flow cells to enable capture and sequencing.

Another example of a DNA and RNA sequencing technique that can be used in the methods of the provided invention is 454 sequencing (Roche) (Margulies, M et al. 2005, Nature, 437, 376-380). 454 sequencing is a sequencing-by-synthesis techonology that utilizes also utilizes pyrosequencing. 454 sequencing of DNA involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed. In another embodiment, pyrosequencing is used to measure gene expression. Pyrosequecing of RNA applies similar to pyrosequencing of DNA, and is accomplished by attaching applications of partial rRNA gene sequencings to microscopic beads and then placing the attachments into individual wells. The attached partial rRNA sequence are then amplified in order to determine the gene expression profile. Sharon Marsh, Pyrosequencing® Protocols in Methods in Molecular Biology, Vol. 373, 15-23 (2007).

Another example of a DNA and RNA detection techniques that may be used in the methods of the provided invention is SOLiD technology (Applied Biosystems). SOLiD technology systems is a ligation based sequencing technology that may utilized to run massively parallel next generation sequencing of both DNA and RNA. In DNA SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In other embodiments, SOLiD Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

Another example of a DNA sequencing technique that may be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is Illumina sequencing, which is a polymerase-based sequence-by-synthesis that may be utilized to amplify DNA or RNA. Illumina sequencing for DNA is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. When using Illumina sequencing to detect RNA the same method applies except RNA fragments are being isolated and amplified in order to determine the RNA expression of the sample.

Another example of a sequencing technology that may be used in the methods of the provided invention includes the single molecule, real-time (SMRT) technology of Pacific Biosciences to sequence both DNA and RNA. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated. In order to sequence RNA, the DNA polymerase is replaced with a with a reverse transcriptase in the ZMW, and the process is followed accordingly.

Another example of a sequencing technique that can be used in the methods of the provided invention is nanopore sequencing (Soni G V and Meller, A Clin Chem 53: 1996-2001) (2007). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using a electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

Additional detection methods can utilize binding to microarrays for subsequent fluorescent or non-fluorescent detection, barcode mass detection using a mass spectrometric methods, detection of emitted radiowaves, detection of scattered light from aligned barcodes, fluorescence detection using quantitative PCR or digital PCR methods.

A comparative genomic hybridization array is a technique for detecting copy number variations within the patient's sample DNA. The sample DNA and a reference DNA are differently labeled using distinct fluorophores, for example, and then hybridized to numerous probes. The fluorescent intensity of the sample and reference is then measured, and the fluorescent intensity ratio is then used to calculate copy number variations. Methods of comparative genomic hybridization array are discussed in more detail in Shinawi M, Cheung S W The array CGH and its clinical applications, Drug Discovery Today 13 (17-18): 760-70.

Another method of detecting DNA molecules, RNA molecules, and copy number is fluorescent in situ hybridization (FISH). In Situ Hybridization Protocols (Ian Darby ed., 2000). FISH is a molecular cytogenetic technique that detects specific chromosomal rearrangements such as mutations in a DNA sequence and copy number variances. A DNA molecule is chemically denatured and separated into two strands. A single stranded probe is then incubated with a denatured strand of the DNA. The signals stranded probe is selected depending target sequence portion and has a high affinity to the complementary sequence portion. Probes may include a repetitive sequence probe, a whole chromosome probe, and locus-specific probes. While incubating, the combined probe and DNA strand are hybridized. The results are then visualized and quantified under a microscope in order to assess any variations.

Commonly used methods known in the art for the quantification of mRNA expression in a sample include northern blotting (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); Beads Array for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, variances in gene expression can also be identified, or confirmed using a microarray techniques, including nylon membrane arrays, microchip arrays and glass slide arrays. Generally, RNA samples are isolated and converted into labeled cDNA via reverse transcription. The labeled cDNA is then hybridized onto either a nylon membrane, microchip, or a glass slide with specific DNA probes from cells or tissues of interest. The hybridized cDNA is then detected and quantified, and the resulting gene expression data may be compared to controls for analysis. The methods of labeling, hybridization, and detection vary depending on whether the microarray support is a nylon membrane, microchip, or glass slide. Nylon membrane arrays are typically hybridized with P-dNTP labeled probes. Glass slide arrays typically involve labeling with two distinct fluorescently labeled nucleotides. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998). In a tissue array, multiple tissue samples are assessed on the same microarray. The arrays allow in situ detection of RNA and protein levels; consecutive sections allow the analysis of multiple samples simultaneously.

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

Comparison of Genetic Characteristics to Respective Controls

Methods of the invention provide for comparing at least two genetic characteristics from a sample to respective controls in order to form a diagnosis of a developmental disorder. In order to determine a disorder diagnosis based on two or more genetic characteristics, the sample's nucleic acid sequence, nucleic acid expression, and nucleic acid copy number are compared to respective controls. The respective controls include reference genetic characteristics obtained from a normal healthy subject, reference genetic characteristics obtained from a subject positively diagnosed with a developmental disorder, and/or reference genetic characteristics associated with known developmental disorders. Depending on the respective control, changes or similarities from the sample genetic characteristics to the respective control are positive indicators for the developmental disorders.

Genetic research has linked autism and other developmental disorders to known variations in nucleic acids including genomic variations at specific chromosomal locations and/or specific genes based on specific nucleic acid sequence mutations, abnormal nucleic acid expression profiles, and copy number variations. The variations at specific chromosomal locations and/or specific genes linked to autism and other developmental disorders are positive indicators for the disorder. Therefore, if a patient's genetic characteristics have the same variations, the patient is diagnosed with disorders corresponding to the variation. Known genetic disorders causally linked to specific genes include but are not limited to an autism spectrum disorder, Aspergers syndrome, Pervasive Developmental Disorder not otherwise specified (atypical autism), Angelman Syndrome, cerebral palsy, Cohen syndrome, Down Syndrome, Fragile X syndrome, IsoDicentric 15, Jacobsen syndrome, Prader-Willi Syndrome, Retts Syndrom, Coffin-Lowry Syndrome, Williams Syndrome, and Cornelia de Lange Syndrome.

Specifically, the above developmental disorders have been linked to variances in DNA sequence, RNA expression, and copy number at the following chromosomal locations: 2p16.3; 2q; 2q37; 5p15; 5p15.2; 5p13.2; 8q22.2; 15q; 15q11-q13; 6q27; 7q; 7q21-22; 7q22; 7q31.1-31.3; 7q31.2; 7q32-36; 7q35-36; pq35-36; pq34; 10q23.2; 10q25; 11q; 11q12-p13; 11q13; 13q21; 14q31; 15q11-13; 16p13.3; 16q24; 17p21; 17q11-17; 17q11.1-q12; 18q21.2-22.3; 19q13; 20p13; 20p13; 21; 21p13; 21q21.2-21.3; 22q11; 22q13; 22q13.3; Xp; Xp11.22-p11.21; Xp22; Xp22.2-p22.1; Xq13.1; Xq22.31-32; Xq27.3; and Xq28. In addition, some of the above chromosomal locations are the location of named genes. Specific genes associated with autism and other developmental disorders include but are not limited to ST7, WNT, CNTNAP2, TSC1, PTEN, NRXN2, NRXN3, TSC2, SLC6A4, APP, SHANK3, NLGN3, NLGN4X, FMR1, MECP2, OCA2, UBE3A, VLDLR, NIOBL, SMC1A, SMC3, VPS13B, CLIP2, ELN, GTF2I, GTF2IRD1, LFMK1, CDKL5, OXTR, CYP11B1, and NTRK1. ST7, WNT, CNTNAP2, TSC1, PTEN, NRXN2, NRXN3, TSC2, SLC6A4, APP, SHANK3, NLGN3, NLGN4X, FMR1, MECP2, OCA2, UBE3A, VLDLR, NIOBL, SMC1A, SMC3, VPS13B, CLIP2, ELN, GTF2I, GTF2IRD1, LFMK1, CDKL5, OXTR, CYP11B1, and NTRK1. Nucleic acid variations indicative of developmental disorders are not limited to the above lists of genes and variances at specific chromosomal locations associated with autism and other developmental disorders because as research progresses more chromosomal locations and variances thereon are being linked to specific developmental disorders.

Methods of the invention provide for assessing a patient nucleic acid sequence for known nucleic acid variants associated with autism or other developmental disorders by comparing the patient's nucleic acid sequence to a control reference sequence. The control reference may include a healthy reference sequence, a reference sequence from a patient positively diagnosed with a developmental disorder, or a reference sequence having known variants linked to autism or other developmental disorders. The mutations may include a missense mutation, a nonsense mutation, an insertion, a deletion, a duplication, a frameshift mutation, a repeated expansion, or any combination thereof. In one embodiment, a patient's sequence is compared directly to a control sequence of a person positively diagnosed with a developmental disorder or a sequences containing mutations known to autism or other developmental disorders. In such embodiment, similarities between the patient's sequence and the control sequence are indicative of a positive diagnosis.

Whereas in other embodiments, the patient's sequence is compared to a normal healthy reference sequence in order to determine abnormal variations in the patient's sequence. The changes between the patient's sequence and normal healthy sequence are then assessed to determine a developmental disorder diagnosis. For example, the abnormal variances are then assessed against known mutations specific to autism and other developmental disorders. If the patient's sequence has the same mutations as those known in developmental disorders, such similar variances represent positive diagnostic markers for the disorder. First, determining the changes in a patient's sequence to a healthy control, and then assessing the changes to known mutations is helpful to assess the patient's sequence to multiple developmental disorder references. It allows one to pinpoint which abnormalities represent a match to each developmental disorder reference being compared.

Methods of the invention provide for assessing for autism based on the patient's nucliec acid expression. Variances in gene expression include differently expressed genes and differential gene expression. A differently expressed gene or differential gene expression refer to a gene whose expression is activated to a higher or lower level in a subject suffering from a disorder, such as an autism spectrum disorder, relative to its expression in a normal or control subject. The a differently expressed gene also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

After detecting gene expression, the patient's sample is compared to a respective control to assess the patient's expression profile in order to form a diagnosis of a developmental disorder. The patient's nucleic acid expression profile may be compared first to a normal nucleic acid expression profile in order to determine differential expressions. The differential expressions in the patent's expression profile are then compared to differential expression patterns of known developmental disorders and similarities thereof are indicative of a positive diagnosis for corresponding developmental disorders. In another embodiment, the patient's nucleic acid expression is compared directly to a gene expression specific to developmental disorders, and similarities between the sample and the specific gene expression are positive markers for the developmental disorders.

Methods of the invention also provide for comparing the nucleic acid copy number of a patient's sample to a control reference. Copy number variants are mutations as compared to a reference sequence such as deletions, amplifications, insertions, and substitutions that affect a segment of DNA that is 1 kilobase or larger. Therefore, copy numbers affect larger number of nucleotides than mutations affecting only a few bases. In one embodiment, changes between the copy number of the patient and the copy number of a healthy control reference sequence may indicate a positive diagnosis of autism or other developmental disorders. After the copy number variants are detected from the normal healthy sequences, the copy number variances are then assessed to known copy number variations specific to autism or other developmental disorders. Similarities between the patient's variants and known copy number variants indicate a positive diagnosis for the developmental disorder. In another embodiment, the copy number of a patient's DNA is compared directly to DNA copy number variants specific to autism or other developmental disorders. Similarities between the patient's copy number variants and copy number variants specific to autism or other developmental disorders indicates a positive diagnosis to the corresponding disorders.

Since the functional consequence of DNA mutations may be difficult to predict, identification of mutations even in known disease risk genes is not a guarantee of disease, and will have a certain false-positive rate when used as a disease predictor. This false-positive can be reduced if the mutation can be confirmed to be de novo, i.e., not present in either parent, by genotyping corresponding loci in the parents, or if shared with a parent who has a (possibly milder) form of the disease.

The false positive rate for inferring disease burden from identified mutations can also be reduced by looking specifically for gene expression changes attributable to identified mutations. We call this “deep integration” of genetic and expression information. For example, if a mutation is expected to result in reduced expression of the mutated gene one could look for confirmation of that reduced expression in the expression data. If the gene's expression is reduced, this provides confirmatory evidence that the mutation has a functional consequence, and therefore strengthens the evidence for disease. If the mutation is predicted to lead to premature termination of the gene's RNA product, then fine-scale expression data such as is produced by RNA sequencing or exon-array methods would predict reduced expression of distal exons, which could be confirmed in expression data. In addition to these direct, or cis effects on the expression on the mutated gene itself, if the mutated gene is a transcription factor, post-translational modifying enzyme (kinase, phosphatase, ligaes, etc.), miRNA or other regulator of gene expression, one would look for indirect, or trans effects: i.e., changes in the expression levels of genes known to be regulated by that regulator. A gene may also influence the expression of other genes indirectly, e.g. via feedback loops, small molecule concentrations, etc. Where the gene is not a known regulator, or the regulatory influences are not known in detail, or are too complex to predict, one would look for derangements of expression in the pathway(s) containing the mutated gene. The combination of cis, trans, and pathway evidence integration helps identify mutations with functional effect on a personalized basis. No single pathway signature that is expected to be common to all individuals with the disorder, instead of variety of risk-gene-associated pathways and subnetworks define independent signatures, any of which can be indicative of disease.

What is claimed is:

1. A method for assessing risk of a cognitive disorder, the method comprising the steps of:
conducting an assay to measure a DNA characteristic known to be associated with a cognitive disorder;
conducting an assay to measure a RNA characteristic known to be associated with a cognitive disorder; and
diagnosing said cognitive disorder based upon said conducting steps.

2. The method of claim 1, wherein said DNA characteristic is selected from a copy number variation, a single nucleotide polymorphism, and a mutation.

3. The method of claim 1, wherein said RNA characteristic is an amount of expressed RNA.

4. The method of claim 1, wherein said cognitive disorder is a developmental cognitive disorder.

5. The method of claim 4, wherein said cognitive developmental disorder is an autism spectrum disorder.

6. The method of claim 5, wherein said autism spectrum disorder is selected from the group consisting of Angelman syndrome, cerebral palsy, Aspergers syndrome, Pervasive Developmental Disorder not otherwise specified (atypical autism), Childhood Disintegrative Disorder, Cohen syndrome, Down syndrome, Fragile X syndrome, IsoDicentric 15, Jacobsen syndrome, Prader-Willi syndrome, Rett syndrome, Coffin-Lowry syndrome, Williams syndrome, and Cornelia de Lange syndrome.

7. The method of claim 1, wherein said conducting steps comprise measuring said DNA characteristic and said RNA characteristic against standards known not to be associated with said cognitive disorder.

8. The method of claim 1, further comprising the step of measuring an amount of a protein in said sample, said protein known to be associated with a cognitive disorder and wherein said diagnosing step is based upon said conducting steps and said measuring step.

9. The method of claim 1, wherein said assay to measure a DNA characteristic comprises sequencing DNA is said sample.

10. A method for classifying a patient suspected of being at risk for a cognitive developmental disorder, the method comprising the steps of:
conducting a first assay to determine at least one genomic change in a sample obtained from a patient;
conducting a second assay to determine a level of RNA expression in said sample from genes known or suspected to be associated with a cognitive developmental disorder; and
classifying said patient as having a cognitive developmental disorder if said genomic change is present and said level of RNA is greater than would be expected in a patient known not to have a cognitive developmental disorder.

11. The method of claim 10, wherein said first conducting step comprises sequencing at least a portion of DNA in said sample.

12. The method of claim 10, wherein said second conducting step comprises measuring a first amount of RNA expressed from a gene known to be associated with a cognitive developmental disorder and comparing said amount with a second amount expressed to be obtained from a sample derived from a patient known not to have an cognitive developmental disorder.

13. The method of claim 12, wherein said second amount is determined empirically.

14. The method of claim 12, wherein said second amount is determined by reference to a computer-generated database.

15. The method of claim 10, wherein said genomic change occurs in a gene selected from the group consisting of ST7, WNT, CNTNAP2, TSC1, PTEN, NRXN2, NRXN3, TSC2, SLC6A4, APP, SHANK3, NLGN3, NLGN4X, FMR1, MECP2, OCA2, UBE3A, VLDLR, NIOBL, SMC1A, SMC3, VPS13B, CLIP2, ELN, GTF2I, GTF2IRD1, LFMK1, CDKL5, OXTR, CYP11B1, and NTRK1. ST7, WNT, CNTNAP2, TSC1, PTEN, NRXN2, NRXN3, TSC2, SLC6A4, APP, SHANK3, NLGN3, NLGN4X, FMR1, MECP2, OCA2, UBE3A, VLDLR, NIOBL, SMC1A, SMC3, VPS13B, CLIP2, ELN, GTF2I, GTF2IRD1, LFMK1, CDKL5, OXTR, CYP11B1, and NTRK1.

16. The method of claim 10, wherein said classifying step comprises determining whether RNA expression from said genes is between about 20% and about 50% greater than that expected to be obtained in a patient known to not have a cognitive developmental disorder.

17. The method of claim 10, wherein said classifying step comprises determining whether RNA expression from said genes is more than about 50% greater than that expected to be obtained in a patient known to not have a cognitive developmental disorder.

18. The method of claim 10, further comprising the step of identifying a disorder based upon said classifying step.

19. The method of claim 18, wherein said disorder is selected from the group consisting of autism spectrum disorders, Angelman syndrome, cerebral palsy, Aspergers syndrome, Pervasive Developmental Disorder not otherwise specified (atypical autism), Childhood Disintegrative Disorder, Cohen syndrome, Down syndrome, Fragile X syndrome, IsoDicentric 15, Jacobsen syndrome, Prader-Willi syndrome, Rett syndrome, Coffin-Lowry syndrome, Williams syndrome, and Cornelia de Lange syndrome.

20. A method for assessing risk of a cognitive developmental disorder, the method comprising the steps of:
obtaining a biological sample from a patient;
determining copy number of one or more genes associated with a cognitive developmental disorder;
measuring RNA expression in said sample;
identifying said patient as at risk for a cognitive developmental disorder if said copy number exceeds a threshold known to be associated with at least one cognitive developmental disorder and said RNA expression exceeds a threshold known to be associated with at least one cognitive developmental disorder.

21. The method of claim 20, wherein said sample is selected from the group consisting of blood, urine, a cheek swab, a skin sample, and hair.

22. The method of claim 20, wherein said identifying step comprises imputing data comprising said copy number and said RNA expression into a computer and utilizing said computer to assess said thresholds.

23. A method for assessing risk of a cognitive developmental disorder, the method comprising the steps of:
determining, in a biological sample, copy number of one or more genes known to be associated with a cognitive developmental disorder;
comparing said copy number with an expected copy number in a sample obtained from a patient having not cognitive developmental disorder;
measuring RNA expression in said sample if there is a statistically-significant difference between said copy number and said expected copy number;
identifying risk of a cognitive developmental disorder if said RNA expression exceeds that which would be expected in a sample obtained from an individual with no cognitive developmental disorder.

24. A method of assessing risk of a cognitive developmental disorder, the method comprising the steps of:
obtaining a first set of copy numbers of a plurality of genes suspected of being associated with a cognitive developmental disorder;
measuring a second set of copy numbers of said genes in a biological sample;
measuring RNA expression in said sample if said second set is statistically-significantly different than said first set; and
assessing risk of a cognitive developmental disorder based upon said measuring steps.

25. A method of assessing risk of a cognitive developmental disorder, the method comprising the steps of:
determining copy number of each of a plurality of genes suspected to be associated with a cognitive developmental disorder;
obtaining expression levels of each of a plurality of RNAs the expression of which is suspected to be associated with a cognitive developmental disorder;
assessing risk of a cognitive developmental disorder based upon a variation in said copy number and said RNA expression relative to a baseline.
